(19) United States
(12) Patent Application Publication
(10) Pub. No.: US 2020/0289300 A1
(43) Pub. Date: Sep. 17, 2020

(54) ASYMMETRICAL STENT WITH RADIALLY VARIABLE PROPERTIES AND METHOD OF USING SAME

(71) Applicant: Cook Medical Technologies LLC, Bloomington, IN (US)

(72) Inventors: Joshua Krieger, Topsfield, MA (US); Alec Cerchiari, Bloomington, IN (US); Jillian Noblet Ivers, Bloomington, IN (US)

(21) Appl. No.: 16/815,152

(22) Filed: Mar. 11, 2020

Related U.S. Application Data
(60) Provisional application No. 62/817,198, filed on Mar. 12, 2019.

(51) Int. Cl. A61F 2/958

(52) U.S. Cl. CPC A61F 2/958; A61F 2002/8483

(57) ABSTRACT

An asymmetrical stent includes a tubular shaped framework with a first region that is exclusive of a second region, both of which extend less than completely around a longitudinal axis. The first region and the second region expand responsive to inflation of a balloon positioned with the tubular shaped framework. The first region is more resistant to expansion than the second region. The tubular shaped framework includes a first plurality of barbs and a second plurality of barbs that all point away from the longitudinal axis. The first and second plurality of barbs being on respective sides of a plane that includes the longitudinal axis and bisects the first region.

### TECHNICAL FIELD

The present disclosure relates generally to stents for use in supporting passageways within a live body, and more particularly to an asymmetrical stent constructed to produce a greater radial outward force in one region than in a second region of the stent.

### BACKGROUND

There exist a number of disease states in which the deployment of a stent is part of accepted clinical protocol. In most of these instances, such as stents used to support partially blocked arteries, the stent is designed to produce substantially uniform outward radial forces around its circumference, so that the supported vessel resumes to a normal circular cross section. In other instances, the disease exhibits an asymmetry such that a uniform radial force stent may be used, but with a less than fully desirable outcome. For instance, in the case of an aortic dissection, the true lumen side and the false lumen side may result in an asymmetry in re-apposing the dissection flap to possibly produce excess stress on the aortic wall. Another example may be May-Thurner syndrome, in which one side of the iliac vein is compressed, while the other side of the vein may be unaffected. In still another example, a tumor or other abnormal tissue growth may tend to push on one side of a body passageway, tending to pinch the passageway closed, whereas the other side of the passageway may be relatively unaffected by the tumor growth.

The present disclosure is directed toward one or more of the problems set forth above.

### SUMMARY

In one aspect, an asymmetrical stent includes a tubular framework that includes a first region exclusive of a second region, both of which extend less than completely around a longitudinal axis. The first region and the second region expand responsive to inflation of a balloon positioned within the tubular shaped framework. The first region is more resistant to expansion than the second region. The tubular shaped framework includes a first plurality of barbs and a second plurality of barbs that all point away from the longitudinal axis. The first and second plurality of barbs are on respective sides of a plane that includes the longitudinal axis and bisects the first region.

In another aspect, a method of using an asymmetrical stent includes positioning the asymmetrical stent at a treatment location. The asymmetrical stent is oriented so that a stiffer region faces a pre-determined portion of a lumen wall of a passageway. The asymmetrical stent is expanded until the asymmetrical stent contacts the lumen wall of the passageway. Then, both the asymmetrical stent and the passageway are further expanded by inflating a balloon. A portion of the asymmetrical stent moves along the lumen wall in a tangential direction perpendicular to a lumen centerline responsive to inflation of the balloon. The balloon is then deflated out of contact with the asymmetrical stent. The passageway and the asymmetrical stent shrink responsive to deflation of the balloon.

### BRIEF DESCRIPTION OF THE DRAWINGS

FIG. 1 is a end view of a stent delivery system at a treatment location in a vessel;

FIG. 2 shows the asymmetrical stent of FIG. 1 self expanded into contact with the vessel wall;

FIG. 3 shows both the asymmetrical stent and vessel being expanded by inflation of a balloon;

FIG. 4 shows the vessel and asymmetrical stent after the balloon of FIG. 3 has been deflated and removed;

FIG. 5 is an enlarged schematic end view of the stent from FIG. 4 showing external barbs and radiopaque markers;

FIG. 6 is a perspective view of the asymmetrical stent from FIGS. 1-5; and

FIG. 7 is a perspective view of an asymmetrical stent according to another aspect of the present disclosure.

### DETAILED DESCRIPTION

The concept of the present disclosure may be described as a stent that exerts different magnitudes of radial force in different regions around its circumference. These regions can then be aligned with desired anatomical locations. This disclosure describes a stent with variable radial stiffness in combination with microbarbs on the luminal surface of the stent. Although the present disclosure is generally applicable to self expanding stents, balloon expanded stents might also find potential applications consistent with the present disclosure. In any event, after expanding into contact with a passageway wall, such as via self expansion, a balloon may be used to over expand the stent and passageway to set the microbarbs, or other friction feature, to focus a greater outward radial force in a desired direction within the passageway.

There are a number of ways in which to vary the radial stiffness of a region of the stent structure. In regard to lattice or structure, stiffness could be modified by the pattern of closed verses open cells, strut length and/or width, strut thickness and/or strut spacing/density. Strut thickness could be modified by changing the thickness of some material, changing materials (nitinol, stainless steel, cobalt chrome, or other suitable material), and/or by grinding, etching, or electro-polishing away some of the material that makes up the stent. Yet another method could be to introduce the presence of a material coating on one region to alter the properties in that region relative to other regions around the circumference of the stent. The concept could also be accomplished by using differential application of heat treatment to the material in each of the different regions such that the final configuration provides variable radial stiffness. A combination of one or more of these strategies could be used to create two or more variable stiffness regions along the circumference of the stent, with the regions extending partially or fully along the axial length of the stent. A configuration of marker bands, such as radiopaque markers, could be used to enable a clinician to ascertain an orientation of the stent in vivo to aide in orienting the stent during a deployment procedure.

Microbarbs are added to the luminal side of the stent structure to oppose the passageway wall. The microbarbs may be arranged in longitudinal bands along the stent surface. The longitudinal band may be as small as a single row of barbs or cover the entire region or even the entire stent. A band of barbs may have a multi-row distribution, be distributed in a field area with no discernable rows, or some combination of both. The barbs may be oriented generally toward a single point on the circumference of the stent, which may be aligned with the anatomical orientation of interest to provide the greatest outward radial stiffness at the convergence.

Upon initial deployment, such as through self expansion, a stent according to the present disclosure will come to an equilibrium against the vessel or passageway wall with approximately uniform radial force along its circumference. In order to redistribute the forces biased against a region of the vessel wall, a balloon dilation of the stent could follow. As the balloon is inflated, the vessel and stent will both elastically expand. The stent will have a biased expansion with the region of low radial stiffness expanding more than the region of higher radial stiffness. As this occurs, the vessel wall will translate (i.e., move) tangentially across the surface of a portion of the stent to achieve an approximately uniform stress. The microbarbs are configured to allow this translation, but engage to prevent reverse translation. Upon deflation of the balloon, the microbarbs stop translation of the vessel over the stent surface back to the initial equilibrium state. As such, the tissue is biased in its loading, with a higher wall stress exerted in the region of greater radial force based upon the shear stress interaction between the barbs and the vessel wall. Those skilled in the art will appreciate that by inflating the balloon to different amounts of expansion, the net bias and radial force may be tuned to a specific application (e.g., re-apposition of a flap against a false lumen wall).

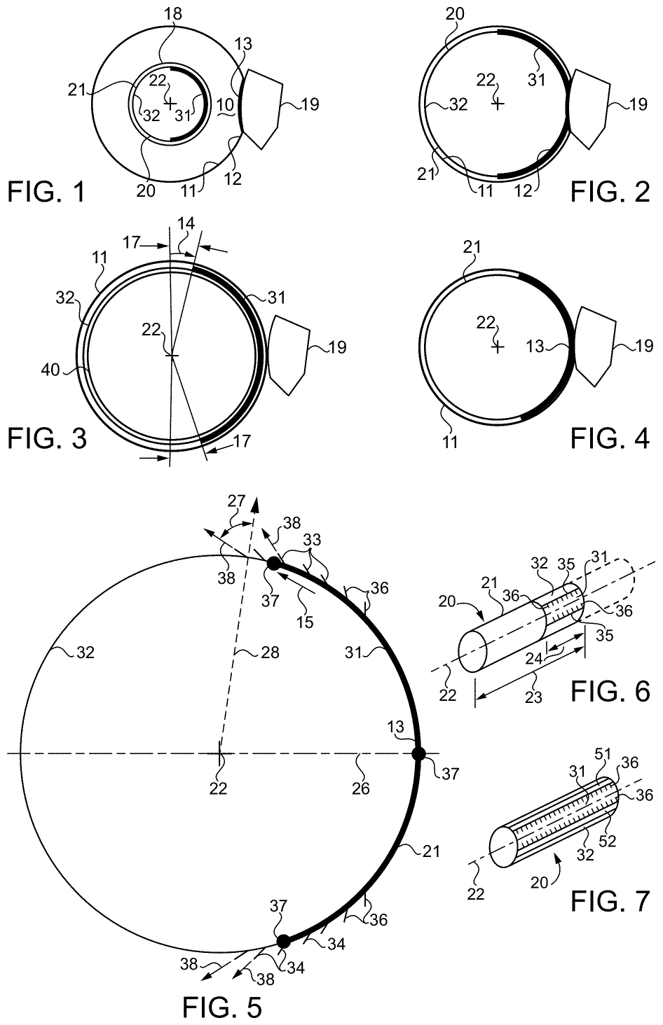

Referring now to FIGS. 1-5, an end view of an asymmetrical stent according to the present disclosure is shown being implanted in a passageway, such as a blood vessel to provide extra radial stiffness and support against one side of the vessel being compressed by some anatomical structure, such as tumor 19. FIG. 1 shows asymmetrical stent 20, which in this example is a self expanding stent, held against expansion by a retractable sheath 18 of a type well known in the art. Asymmetrical stent 20 includes a first region 31 of higher radial stiffness and a second region 32 with a lower radial stiffness relative to the first region 31. The radial stiffness within each region need not be uniform and may vary. These relative stiffnesses are shown schematically by the first region 31 having a thicker profile than the second region 32. This difference is for illustrative purposes only, as the two regions 31 and 32 may have virtually indistinguishable thicknesses when viewed in an end-wise profile view, but could have different thicknesses without departing from the present disclosure. Asymmetrical stent 20 generally, and the first and second regions 31, 32 specifically, form a tubular shaped framework of a type well known in the art and not taught again here. In this example, the asymmetrical stent 20 is intended to be deployed in a manner that produces an extra radial outward force at treatment location 10 against the pressing tumor 19 growth outside of passageway 11, which may be an artery, a vein or any other passageway in a living body in need of support by a stent. An enlarged view of the asymmetrical stent 20 is shown in FIG. 5, which includes rows of microbarbs 36, that are omitted for clarity from the illustrations of FIGS. 1-4.

FIG. 2 shows asymmetrical stent 20 after self expanding into contact with the lumen wall 12 of vessel 11 responsive to movement release by the retractable sheath (FIG. 1). If the procedure was stopped at this point, stent 20 may behave in passageway 11 similar to self expanding stents well known in the art in that the lumen wall 12 will be substantially supported with a nearly uniform radial outward force. The asymmetrical capability of stent 20 according to the present disclosure is activated by expanding both the stent 20 and the passageway 11 beyond the equilibrium point shown in FIG. 2 with a balloon. When this occurs as shown in FIG. 3, the fact the first region 31 is more resistant to expansion than the second region 32 results in a portion of the stent 20 in the region where the first and second regions 31, 32 meet to move the stent along the lumen wall 12 in a tangential direction 14. The magnitude 17 of the movement in tangential direction 14 is proportional to an inflation pressure in balloon 40. Thus, those skilled in the art will appreciate that the magnitude 17 can be calibrated to the inflation pressure of balloon 40 so that the clinician may determine magnitude 17 based upon expected calibration data that could be determined experimentally. Thereafter as shown in FIG. 4, balloon 40 is deflated and both asymmetrical stent 20 and passageway 11 return to a new equilibrium that is similar in size or diameter to the equilibrium reflected in FIG. 2 but with a different balance of forces such that the first region 31 places an extra radial outward force at the predetermined location 13 adjacent to tumor 19, which is a greater radial outward force on the lumen wall 12 than by second region 32 directly opposite of location 13. Although the residual displacement magnitude 17 may be equal above and below plane 25 as shown in FIGS. 4 and 5, the residuals may be different.

As best shown in FIG. 5, when balloon 40 is deflated, the first and second plurality of barbs 33, 34 engage the lumen wall 12 and prevent the asymmetrical stent 20 from moving in a reverse tangential direction 15 back toward the equilibrium position illustrated by FIG. 2. The microbarbs shown in FIG. 5 are omitted from the views of FIGS. 1-4 for clarity. Those skilled in the art will appreciate that a first plurality of barbs 33 and a second plurality of barbs 34 point generally away from the location 13 where the extra radial outward force acts, and to a lessor extent point away from longitudinal axis 22. The orientation of each of the barbs 33, 34 point in a direction that is at an acute angle 27 with respect to a radius vector 28 that extends from longitudinal axis 22 through the respective barb 33, 34. Those skilled in the art will appreciate that the acute angle 27 may vary for the barbs as they are located closer or further away from the location 13 where the extra radial outward force is needed.

Referring now in addition to FIGS. 6 and 7, the first region 31 and the second region 32 may be contiguous with one another as shown in the embodiment of FIGS. 1-5. However, in order to provide a less abrupt change between the increased stiffness of the first region relative to that of the second region 32, the first and second regions 31, 32 may be separated by first and second transition regions 51, 52, respectively as best shown in FIG. 7. FIG. 7 shows an asymmetrical stent 120 in which the first and second regions 31 and 32 extend the full length of asymmetrical stent 120. However, both regions 31 and 32 are separated by transition regions 51 and 52 so that the change in stiffness from that of the first region 31 to that of the second region 32 occurs in a more continuous and less abrupt manner than that associated with the embodiment of FIGS. 1-5. FIG. 6 shows that the asymmetrical portion of asymmetrical stent 20 may have a length 24 that is less than the full length 23 of the asymmetrical stent 20 without departing from the scope of the present disclosure. Thus, an asymmetrical stent 20 according to the present disclosure may include a segment that has substantially uniform radial outward bias around its circumference, such as that associated with typical well known stents, but include a length portion 24 that includes the asymmetric features associated with that of the present disclosure. Furthermore, an asymmetrical stent 20 according to the present disclosure may include an asymmetric segment 24 that is sandwiched between conventional stent tubular frameworks on opposite ends as shown by the solid end dotted lines in FIG. 6.

All versions of an asymmetrical stent according to the present disclosure include a tubular shaped framework 21 that defines a longitudinal axis 22. The tubular shaped framework 21 includes a first region 31 that exclusive of a second region 32, both of which extend less than completely around the longitudinal axis 22. The first region 31 and the second region 32 expand responsive to inflation of a balloon 40 positioned within the tubular shaped framework 21. Those skilled in the art will appreciate that asymmetrical stent may self expand to assume a configuration that allows a balloon 40 to be positioned within the tubular shaped framework 21. The first region 31 is more resistant to expansion than the second region 32. The tubular shaped framework 21 also includes a first plurality of barbs 33 and a second plurality of barbs 34 that all point away from the longitudinal axis 22. The first and second plurality of barbs 33, 34 are on respective sides of a plane 26 that include the longitudinal axis 22 and bisects the first region 31, as best shown in FIG. 5. Although the first region 31 may be contiguous to the second region 32, such as at two locations around the circumference of asymmetrical stent 20, the two regions may be separated by a first transition region 51 and a second transition region 52, as best shown in FIG. 7, in order to provide a more continuous transition between the different stiffness levels of the first and second regions 31, 32 respectively. Inclusion of transition regions 51 and 52 may be desirable in order to reduce the potential adverse effects from fatigue that the asymmetrical stent 20 may encounter during its working life. Although asymmetrical stent 20 is preferably self expanding in order to self expand to reach the equilibrium configuration associated with FIG. 2, those skilled in the art will appreciate that the stent may be balloon expanded, but still exhibit elasticity when over expanded by a balloon as shown in FIG. 3. The first region 31 may be contiguous with the second region 32 at two longitudinal lines 35 such that the first region 31 and the second region 32 together completely encircle the longitudinal axis 22. In the case of the embodiment shown in FIG. 7, the first and second regions 31, 32 are not contiguous and are separated by transition regions 51 and 52 so that together the first region 31, the second region 32, the first transition region 51 and the second transition region 52 together completely encircle the longitudinal axis 22. Although the first region 31 and likely the second region 32, may extend the full length of the tubular shaped framework 21, this disclosure also contemplates asymmetrical stents 20 in which the first region 31 extends less than a length 23 of the stent along longitudinal axis 22, as best shown in FIG. 6. Although the first and second regions 31, 32 may have equal areas when in the equilibrium configuration associated with FIG. 2, one of the first region 31 and the second region 32 may have a smaller area than the other of the first and second regions 31, 32. FIG. 6 also shows a configuration in which the first region 31 has a smaller area than the second region 32. The first and second plurality of barbs 33 and 34 may include at least one row of barbs 36 that extend along longitudinal axis 22 for a length on tubular shaped framework 21 that is about equal to the respective region 31 or 32. Although not shown, the asymmetrical shape 20 may also include barbs in the non-asymmetric portion that flanks one or both sides of the asymmetrical first and second regions 31 and 32 as shown in FIG. 6. Preferably, the stent may include a plurality of rows of barbs 36 on opposite sides of the plane 26 that bisects the first region as shown in FIG. 5. Thus, the first and second plurality of barbs 33, 34 may include a band of barbs extending along the longitudinal axis 22 that is comprised or two or more rows of barbs 36, or a field of barbs with no discernable rows. Also as best shown in 5, in order to focus the excess radial outward force at the predetermined location 13 on the outer surface of asymmetrical stent 20, the first plurality of barbs 33 may point in directions that diverge from the pointing directions 38 associated with the second plurality of barbs 34. Finally, in order to assist the clinician in properly orienting the stent during an implantation procedure so that the predetermined location 13 is oriented properly within the patient passageway, the asymmetrical stent 20 may include a plurality of radiopaque markers 37 that are attached to the tubular shaped framework in a pattern that identifies an orientation of the first region 31 about the longitudinal axis 22 with imaging techniques well known in the art.

### INDUSTRIAL APPLICABILITY

The present disclosure finds general applicability to situations in which a passageway within a patient can benefit from asymmetric internal support so that more radial force is applied at one location of the lumen wall of the passageway verses a radial outward support opposite to that location. These circumstances might occur, for instance, when some other condition, such as a tumor, is pressing against the outside of a passageway on one side of the passage but not the other, so that excess radial outward force is necessary to push against the pinching tumor or other phenomenon. Other potential applications include supporting a vessel in a iliac vein responsive to May-Thurner syndrome. Although the present disclosure finds general applicability in any body passageway, the present disclosure finds particular application to supporting blood vessels, including both veins and arteries in the need of some asymmetrical support afforded by the asymmetrical stent of the present disclosure.

Referring again to FIGS. 1-4, a method of using an asymmetrical stent 20 according to the present disclosure includes positioning the asymmetrical stent 20 at a treatment location 10 as shown in FIG. 1. Passageway may be in a live body, or be an artificial passageway for teaching or demonstration purposes. Next, the asymmetrical stent is oriented so that a stiffer region 31 faces a predetermined portion 13 of a lumen wall 12 of a passageway 11. This may be accomplished by the clinician appropriately rotating a delivery system while observing radiopaque markers 37 in a conventional manner. Next, the asymmetrical stent 20 is expanded until the stent 20 contacts the lumen wall 12 of the passageway 11. This may be accomplished by withdrawing containment sheath 18 as shown in FIG. 1 to allow the asymmetrical stent to self expand out into contact with the lumen wall 12 as shown in FIG. 2 until achieving an equilibrium. Next, both the asymmetrical stent 20 and the passageway 11 are further expanded by inflating a balloon 40 as shown in FIG. 3. When this occurs, portions of the asymmetrical stent 20 move along the lumen wall 12 in a tangential direction 14 because the first region 31 is more resistant to expansion than the second region 32. Next, the balloon 40 is deflated and moved out of contact with the asymmetrical stent shown in FIG. 4. The passageway 11 and the asymmetrical stent 20 elastically shrink back from the over expanded configuration of FIG. 3 responsive to deflation of the balloon 40, as shown in FIG. 4. However, barbs, or some other friction feature, that are attached to the tubular shaped framework 21 of the asymmetrical stent 20, or incorporated onto a graft fabric, inhibit reverse movement of the asymmetrical stent 20 along the lumen wall 12 in a reverse tangential direction 15 responsive to deflation of the balloon. This phenomenon results in the engagement of the barbs with the passageway wall 12 in a shear stress interaction concentrating excess radial outward force at the predetermined location 13 that is located at about the center point of the first region 31. Thus, the first and second plurality of barbs assist in directing a radially outward force from the stent 20 toward the predetermined portion 13 of the lumen wall 12 that is greater than a radially outward force on the lumen wall opposite the predetermined portion 13. Those skilled in the art will appreciate that the distance of tangential movement 14 is proportional to the amount that the vessel or passageway 11 and 20 are overexpanded by balloon 40, whose diameter is primarily responsive to the inflation pressure within the balloon. Thus, one may set a magnitude of the radially outward force at location 13 responsive to a predetermined inflation pressure of the balloon 40. In addition, one may inflate the balloon 40 to a certain magnitude and ascertain whether the asymmetrical force is adequate. If not, the balloon may be re-inflated to a greater pressure to increase the asymmetrical force at the predetermined location 13. Those skilled in the art will appreciate that because the movement is in one direction, one may not decrease the excess radial force. Thus, the clinician may step wise re-inflate the balloon to incrementally greater pressures until the asymmetrical stent 20 is properly configured with a desired radially outward force magnitude.

The present description is for illustrative purposes only, and should not be construed to narrow the breadth of the present disclosure in any way. Thus, those skilled in the art will appreciate that various modification might be made to the presently disclosed embodiments without departing from the full and fair scope and spirit of the present disclosure. Other aspects, features and advantages will be apparent upon an examination of the attached drawings and appended claims.

What is claimed is:

1. An asymmetrical stent comprising:
a tubular shaped framework that defines a longitudinal axis;
the tubular shaped framework including a first region exclusive of a second region, both of which extend less than completely around the longitudinal axis;
wherein the first region and the second region expand responsive to inflation of a balloon positioned within the tubular shaped framework;
wherein the first region is more resistant to expansion than the second region;
wherein the tubular shape framework includes a first plurality of barbs and a second plurality of barbs that all point away from the longitudinal axis; and
the first and second plurality of barbs being on respective sides of a plane that includes the longitudinal axis and bisects the first region.

2. The asymmetrical stent of claim 1 wherein the first region is contiguous with the second region.

3. The asymmetrical stent of claim 2 wherein the first region is contiguous with the second region at two longitudinal lines such that the first region and the second region together completely encircle the longitudinal axis.

4. The asymmetrical stent of claim 1 wherein the first region extends less than a length of the stent along the longitudinal axis.

5. The asymmetrical stent of claim 1 wherein the first region has a smaller area than the second region.

6. The asymmetrical stent of claim 1 wherein each of the first plurality of barbs and the second plurality of barbs includes at least one row of barbs extending along the longitudinal axis.

7. The asymmetrical stent of claim 1 wherein each of the first plurality of barbs and the second plurality of barbs includes a band of barbs extending along the longitudinal axis.

8. The asymmetrical stent of claim 1 wherein each barb of the first plurality of barbs and the second plurality of barbs points in a direction that is at an acute angle with respect to a respective radius vector that extends from the longitudinal axis through the respective barb.

9. The asymmetrical stent of claim 8 wherein the first plurality of barbs point in directions that diverge from pointing directions of the second plurality of barbs.

10. The asymmetrical stent of claim 1 including a plurality of radiopaque markers attached to the stent in a pattern that identifies an orientation of the first region about the longitudinal axis.

11. The asymmetrical stent of claim 1 wherein the stent is a self-expanding stent.

12. The asymmetrical stent of claim 1 wherein the first region is separated from the second region by a first transition region and a second transition region.

13. A method of using an asymmetrical stent comprising the steps of:
positioning the asymmetrical stent at a treatment location;
orienting the asymmetrical stent so that a stiffer region faces a predetermined portion of a lumen wall of a passageway;
expanding the asymmetrical stent until the asymmetrical stent contacts the lumen wall of the passageway;
expanding both the asymmetrical stent and the passageway by inflating a balloon;
moving the asymmetrical stent along the lumen wall in a tangential direction perpendicular to a lumen centerline responsive to inflation of the balloon;
deflating the balloon out of contact with the asymmetrical stent; and
wherein the passageway and the asymmetrical stent shrink responsive to deflation of the balloon.

14. The method of claim 13 wherein a first plurality of barbs and a second plurality of barbs inhibit reverse movement of the asymmetrical stent with along the lumen wall in a reverse tangential direction responsive to deflation of the balloon.

15. The method of claim 13 including orienting the first and second plurality of barbs to direct a radially outward force from the asymmetrical stent toward the predetermined portion of the lumen wall that is greater than a radially outward force on the lumen wall opposite to the predetermined portion.

16. The method of claim 15 including setting a magnitude of the radially outward force responsive to an inflation pressure of the balloon.

17. The method of claim 13 wherein the orienting step includes determining an orientation of stent markers about a vessel centerline.

18. The method of claim 13 wherein the step of expanding the asymmetrical stent until the asymmetrical stent contacts the lumen wall of the passageway is accomplished by unsheathing a self-expanding stent.
